SEQUENCE LISTING (12 sequences)

SEQ ID NO: 1 - protein, 12 aa, synthetic construct
KHLHYHSSVRYG

SEQ ID NO: 2 - protein, 35 aa, synthetic construct
QDCQNAHQEFRFWPFLVIIVILSALFLGTLACFCV

SEQ ID NO: 3 - protein, 120 aa, synthetic construct
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 4 - protein, 51 aa, synthetic construct
MGWIRGRRSRHSWEMSEFHNYNLDLKKSDFSTRWQKQRCPVVKSKCRENAS

SEQ ID NO: 5 - protein, 316 aa, synthetic construct
MALPVTALLLPLALLLHAARPKHLHYHSSVRYGGGGSKHLHYHSSVRYGGGGSKHLHYHSSVRYGTTTPAPRPPTPAPTIASQPLSLRPEACRPAAGGAVHTRGLDFACDQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSMGWIRGRRSRHSWEMSEFHNYNLDLKKSDFSTRWQKQRCPVVKSKCRENAS

SEQ ID NO: 6 - DNA, 36 bp, synthetic construct
  source 1..36: mol_type = other DNA
aagcacctgcactaccacagcagcgtgagatacggc

SEQ ID NO: 7 - DNA, 105 bp, synthetic construct
  source 1..105: mol_type = other DNA
caggactgtcagaatgcccatcaggaattcagattttggccgtttttggtgatcatcgtgattctaagcgcactgttccttggcacccttgcctgcttctgtgtg

SEQ ID NO: 8 - DNA, 360 bp, synthetic construct
  source 1..360: mol_type = other DNA
tggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaaccagcatatacattatattcattaattcagccttccaggaagtctggttccaggaagaggaaccacagcccttccttcaatagcactatctatgaagtgattggaaagagtcaacctaaagcccagaaccctgctcgattgagccgcaaagagctggagaactttgatgtttattcc

SEQ ID NO: 9 - DNA, 153 bp, synthetic construct
  source 1..153: mol_type = other DNA
atggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatct

SEQ ID NO: 10 - DNA, 951 bp, synthetic construct
  source 1..951: mol_type = other DNA
atggccttaccagtgaccgccttgctcctgccgctggccttgctgctccacgccgccaggccgaagcacctgcactaccacagcagcgtgagatacggcggcggcggcagcaagcacctgcactaccacagcagcgtgagatacggcggcggcggcagcaagcacctgcactaccacagcagcgtgagatacggcaccactaccccagcaccgaggccacccaccccggctcctaccatcgcctcccagcctctgtccctgcgtccggaggcatgtagacccgcagctggtggggccgtgcatacccggggtcttgacttcgcctgcgatcaggactgtcagaatgcccatcaggaattcagattttggccgtttttggtgatcatcgtgattctaagcgcactgttccttggcacccttgcctgcttctgtgtgtggaggagaaagaggaaggagaagcagtcagagaccagtcccaaggaatttttgacaatttacgaagatgtcaaggatctgaaaaccaggagaaatcacgagcaggagcagacttttcctggaggggggagcaccatctactctatgatccagtcccagtcttctgctcccacgtcacaagaaccagcatatacattatattcattaattcagccttccaggaagtctggttccaggaagaggaaccacagcccttccttcaatagcactatctatgaagtgattggaaagagtcaacctaaagcccagaaccctgctcgattgagccgcaaagagctggagaactttgatgtttattccatggggtggattcgtggtcggaggtctcgacacagctgggagatgagtgaatttcataattataacttggatctgaagaagagtgatttttcaacacgatggcaaaagcaaagatgtccagtagtcaaaagcaaatgtagagaaaatgcatcttaa

SEQ ID NO: 11 - protein, 45 aa, synthetic construct
TTTPAPRPPTPAPTIASQPLSLRPEACRPAAGGAVHTRGLDFACD

SEQ ID NO: 12 - DNA, 135 bp, synthetic construct
  source 1..135: mol_type = other DNA
accactaccccagcaccgaggccacccaccccggctcctaccatcgcctcccagcctctgtccctgcgtccggaggcatgtagacccgcagctggtggggccgtgcatacccggggtcttgacttcgcctgcgat

(19) United States
(12) Patent Application Publication
(10) Pub. No.: US 2024/0076315 A1
(43) Pub. Date: Mar. 7, 2024

(54) KHL POLYPEPTIDE, AND USE THEREOF IN PREPARATION OF TABP-EIC CELL

(71) Applicant: ALLIFE MEDICINE (ZHUHAI) LIMITED, Hengqin New District (CN)

(72) Inventors: Le YIN, Hengqin New District (CN); Yuchun GU, Hengqin New District (CN)

(21) Appl. No.: 18/380,834

(22) Filed: Oct. 17, 2023

Related U.S. Application Data
(63) Continuation of application No. PCT/CN2022/090277, filed on Apr. 29, 2022.

(30) Foreign Application Priority Data
Jul. 14, 2021 (CN) 202110797551.5

(51) Int. Cl. C07K 7/08; A61K 39/00; A61P 35/00; C07K 14/705; C12N 5/0783; G01N 33/574

(52) U.S. Cl. CPC C07K 7/08; A61K 39/4613; A61K 39/4631; A61K 39/464495; A61P 35/00; C07K 14/70503; C07K 14/70517; C07K 14/7056; C12N 5/0646; G01N 33/57434; A61K 2239/15; C07K 2319/02; C07K 2319/03

(57) ABSTRACT

Provided in the present disclosure are a KHL polypeptide, and the use thereof in the preparation of a TABP-EIC cell. In addition, also provided in the present disclosure are a KHL polypeptide conjugate, a tumor-antigen-binding polypeptide containing the KHL polypeptide, a DNA molecule, a carrier, a host cell and a pharmaceutical composition. The tumor-antigen-binding polypeptide is composed of the KHL polypeptide, a transmembrane domain and/or a signal transduction domain.

### CROSS-REFERENCE TO RELATED APPLICATION(S)

This present application is a continuation application of International Patent Application No. PCT/CN2022/090277, filed on Apr. 29, 2022, which claims priority to Chinese Patent Application No. 202110797551.5, filed on Jul. 14, 2021, the disclosures of which are hereby incorporated by reference in their entireties.

### TECHNICAL FIELD

The present disclosure relates to the field of biotechnology, in particular to a KHL peptide and its application in the preparation of TABP-EIC cells.

### BACKGROUND

Tumor chimeric antigen receptor (CAR) therapy refers to a new type of precise targeted therapy that has achieved good results in clinical tumor treatment through optimization and improvement in recent years. It is a very promising new tumor immunotherapy method that may cure cancer. Usually, CAR activates immune cells through genetic engineering technology and installs a localization navigation device CAR. Ordinary T cells and NK cells are correspondingly recombined into CAR-T cells and CAR-NK cells. CAR-T cells and CAR-NK cells can use their chimeric CAR to specifically recognize tumor cells in the body and release a large number of effector factors through immune action, which can efficiently kill tumor cells and achieve the goal of treating malignant tumors. In addition, transplanting immune cells targeted for CAR back into the original patient's body can avoid the challenge of attacking the autoimmune system.

Natural killer (NK) cells are an important component of the non-specific immune system and a key mediator of innate immune system responses. NK cells are a broad-spectrum immune cell with the specific function of quickly detecting and destroying abnormal cells (such as cancer or virus infected cells), and can demonstrate strong activity of dissolving abnormal cells without the need for early sensitization or HLA typing. But the number of NK cells is relatively small, accounting for only 10%-15% of lymphocytes in peripheral blood, and the content of NK cells in umbilical cord blood is lower, only about 5%. The content of NK cells in normal human peripheral blood and umbilical cord blood is far from meeting the needs of clinical treatment. The number and purity of NK cells are important factors affecting clinical efficacy. If the number of NK cells is too small, it cannot achieve therapeutic effects. If the purity is low, it will affect the killing effect on tumors.

Chimeric antigen receptor (CAR) modified immune cells use genetic engineering methods to modify immune cells to express exogenous anti-tumor genes. The CAR gene mainly includes the extracellular recognition domain and the intracellular signal transduction domain: the former is used to recognize tumor surface specific molecules, and the latter is used to initiate the immune cell response after recognizing tumor surface molecules, playing a cytotoxic role.

NK cells modified by CAR structure can efficiently recognize tumor cells and kill them by releasing killing mediators, inducing target cell apoptosis, and other means. However, the existing technology for preparing CAR-NK cells has the problem of complex preparation methods, insufficient cell activity, and limited quantity.

The present disclosure screened KHL peptides with better tumor specific binding efficiency through phage display technology, and verified the specificity of the peptides. After binding with detectable markers, KHL peptides can be used for the detection of cancer marker PSMA. TABP-EIC (Tumor Antigen Binding Peptide Engineering Immune Cell) cells prepared from KHL peptides can accurately target cells expressing PSMA.

### SUMMARY

The present disclosure provides a KHL peptide that specifically binds to PSMA (Prostate Specific Membrane Antigen), its conjugate, its tumor antigen binding peptide, DNA molecule, carrier, and host cell.

Polypeptide

On the one hand, the present disclosure provides a KHL peptide that specifically binds to PSMA, the KHL peptide is selected from any of the following:
1) a peptide shown in SEQ ID NO:1;
2) a peptide with PSMA specific binding function formed by replacing, missing, or adding one or more (2, 3, 4, 5, 6) amino acid residues to the peptide shown in SEQ ID NO:1;
3) peptides with over 90% homology to the peptide shown in SEQ ID NO:1, specifically, the peptide has 90%, 91%, 92%, 93%, 94%, 95%, 96%, 97%, 98%, and 99% homology to SEQ ID NO: 1.

Preferably, the KHL peptide is a peptide shown in SEQ ID NO: 1.

Preferably, the KHL peptide can contain one or more synthesized amino acids, which are known in the art and include but are not limited to amino cyclohexanolic acid, n-leucine α-Amino n-decanoic acid, homoserine, S-acetylaminomethyl cysteine, trans-3- and trans-4-hydroxyproline, 4-aminophenylalanine, 4-nitrophenylalanine, 4-chlorophenylalanine, 4-carboxylphenylalanine β-Phenylserine β-Hydroxyphenylalanine, phenylglycine α-Naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl lysine, N′, N′-dibenzyl lysine, 6-hydroxylysine, guanine α-Aminocyclopentane carboxylic acid α-Aminocyclohexane carboxylic acid α-Aminocyclohexane carboxylic acid α-(2-Amino-2-norcamphene)-carboxylic acid α,γ-Diaminobutyric acid α,β-Diaminopropionic acid, homophenylalanine, and α-Tert butyl glycine.

Conjugate

On the other hand, the present disclosure provides a conjugate of KHL peptides specifically binding to PSMA.

Preferably, the conjugate of the KHL peptide includes the KHL peptide and a detectable marker.

Preferably, the detectable markers include fluorescent dyes, fluorescent molecules, chemiluminescent markers, dye molecules, phosphorescent molecules, biotins, radioactive isotopes, molecules that can absorb in the UV spectrum, molecules that can absorb in near-infrared radiation, or molecules that can absorb in far-infrared radiation.

Preferably, the fluorescent dyes include but are not limited to rhodamine, p-methylaminophenol, fluorescein, thiofluorescein, aminofluorescein, carboxyfluorescein, chlorofluorescein, methylfluorescein, sulfonylfluorescein, aminop-methylaminophenol, carboxyl-p-methylaminophenol, chloro-p-methylaminophenol, methyl-p-methylaminophenol, sulfon-p-methylaminophenol, aminorhodamine, carboxyl rhodamine, chlororhodamine, methyl rhodamine Sulfo rhodamine, as well as thio rhodamine, cyanine, indole carbon cyanine, oxacarbocyanine, thiacyanine, anthocyanin, cyanine dyes (such as cyanine 2, cyanine 3, cyanine 3.5, cyanine 5, cyanine 5.5, cyanine 7), oxadiazole derivatives, pyridoxazole, nitrobenzoxazole, benzonitrobenzene, pyrene derivatives, waterfall blue, oxazine derivatives, Nile red, Nile blue, cresol violet, oxazine 170, acridine derivatives, prodrome, acridine orange, acridine yellow Aromatic methyl derivatives, gold amines, thiamine dyes, sulfonated thiamine dyes, AlexaFluor (such as Alexa fluorescence 594, Alexa fluorescence 633, Alexa fluorescence 647, Alexa fluorescence 700), crystal violet, malachite green, tetrapyrrole derivatives, porphyrins, phthalocyanines, bilirubin, Cy5.5, indocyanine green (ICG), DyLight750, or IRdye800.

Preferably, the fluorescent molecules include but are not limited to FAM, FITC, VIC, JOE, TET, CY3, CYS, ROX, Texas Red, or LC RED460.

Preferably, the chemiluminescent markers include, but are not limited to, peroxidase, alkaline phosphatase, luciferase, jellyfish luminescent protein, functionalized iron porphyrin derivatives, luminol, luminol, acridine ester, sulfonamide, etc.

Preferably, the luciferase includes, but is not limited to, the luciferase of the long bellied water flea (Gaussia), the luciferase of the sea kidney (Renilla), the luciferase of the lumbar flagellate, the luciferase of the firefly, the luciferase of the fungus, the luciferase of the bacteria, and the luciferase of the vargula.

Preferably, the detectable marker is a fluorescent molecule.

Preferably, the fluorescent molecule is FITC.

Tumor Antigen Binding Peptide

On the other hand, the present disclosure provides a tumor antigen binding peptide, which comprises a specific binding domain specifically binding to PSMA.

Preferably, the specific binding domain includes the aforementioned KHL peptide.

Preferably, the specific binding domain is multiple replicates of the aforementioned KHL peptide.

Preferably, the multiple replicates are 1-10; specifically, once, twice, three times, four times, five times, six times, seven times, eight times, nine times, ten times.

Preferably, the specific binding domain is three replicates of the aforementioned KHL peptide.

Preferably, the multiple replicates of the KHL peptide are connected by linkers.

Preferably, the linker is GS, GGS, or GGGS.

Preferably, the linker is GGGS.

Preferably, the tumor antigen binding peptide further includes a hinge area, a transmembrane domain, and/or a signal transduction domain.

Preferably, the hinge area includes one or more combinations of CD8a hinge area, CD28 hinge area, CD4 hinge area, CD5 hinge area, CD134 hinge area, CD137 hinge area, and ICOS hinge area.

Preferably, the hinge area selects CD8 a hinge area.

Preferably, the amino acid sequence of the hinge area is shown in SEQ ID NO: 11.

Preferably, the nucleic acid sequence of the hinge area is shown in SEQ ID NO: 12.

Preferably, the transmembrane domain includes a transmembrane domain of a protein, which includes the transmembrane domain of the 2B4 gene and α, β or ζ chain of the T cell receptor, CD28, CD3 ε, CD45, CD4, CD5, CD8, CD9, CD16, CD22, CD33, CD37, CD64, CD80, CD86, CD123, CD134, CD137, and CD154.

Preferably, the transmembrane domain selects the transmembrane region of the 2B4 gene.

Preferably, the amino acid sequence of the transmembrane domain is shown in SEQ ID NO: 2.

Preferably, the nucleic acid sequence of the transmembrane domain is shown in SEQ ID NO: 7.

Preferably, the signal transduction domain includes a co stimulus domain and/or a primary signal transduction domain.

Preferably, the co stimulus domain includes functional signaling domains of 2B4, CD3 ζ, OX40, CD2, CD27, CD28, CDS, ICAM-1, LFA-1 (CD11a/CD18), ICOS (CD278), and 4-1BB (CD137).

Preferably, the co stimulus signal selects an intracellular signal transduction structure of the 2B4 gene.

Preferably, the amino acid sequence of the co stimulus signal is shown in SEQ ID NO: 3.

Preferably, the nucleic acid sequence of the co stimulus signal is shown in SEQ ID NO: 8.

Preferably, the primary signal transduction domain selects the intracellular signal transduction structure of the NKG2D gene;

Preferably, the amino acid sequence of the primary signal transduction domain is shown in SEQ ID NO: 4.

Preferably, the nucleic acid sequence of the primary signal transduction domain is shown in SEQ ID NO: 9.

Preferably, the sequence of the tumor antigen binding peptide is KHL peptide-transmembrane domain—co stimulatory domain—primary signal transduction domain.

Preferably, the amino acid sequence of the tumor antigen binding peptide is at positions 22-316 as shown in SEQ ID NO:5.

Preferably, the tumor antigen binding peptide can also be connected to the signal peptide.

Preferably, the amino acid sequence of the tumor antigen binding peptide connected to the signal peptide is shown in SEQ ID NO:5.

DNA Molecules

On the other hand, the present disclosure provides a DNA molecule encoding the aforementioned KHL peptide.

Preferably, the sequence of the DNA molecule encoding the KHL peptide is shown in SEQ ID NO: 6.

On the other hand, the present disclosure provides a DNA molecule encoding the aforementioned tumor antigen binding peptide.

Preferably, the sequence of the DNA molecule encoding the tumor antigen binding peptide according to claim 2 is the 64-948 positions of SEQ ID NO: 10.

Preferably, the tumor antigen binding peptide can also be connected to the signal peptide.

Preferably, the sequence of the DNA molecule of the tumor antigen binding peptide connected to the signal peptide is shown in SEQ ID NO: 10.

Carrier

On the other hand, the present disclosure provides a carrier comprising DNA molecules encoding peptides and/or DNA molecules encoding tumor antigen binding peptides.

Preferably, the carrier includes plasmids (expression plasmids, cloning vectors, small loops, microcarriers, double micro chromosomes), lentiviral vectors, adenoviral vectors, or retroviral vectors.

Preferably, the lentivirus vector includes a primate recombinant lentivirus vector, namely a recombinant human immunodeficiency virus (HIV) or a recombinant simian immunodeficiency virus (SIV).

Preferably, the lentivirus vector includes non primate recombinant lentivirus vectors, namely recombinant equine infectious anemia virus (EIAV), recombinant feline immunodeficiency virus (Hy), or recombinant caprine arthritis encephalitis virus (CAEV).

Preferably, the lentivirus vector comprises the following: pLK0.1 puro, pLK0.1 CMV tGFP, pLK0.1 puro CMV tGFP, pLK0.1 CMV Neo, pLK0.1 Neo, pLK0.1 Neo CMV tGFP, pLK0.1 puro CMV TagCFP, pLK0.1 puro CMV TagYFP, pLKO. 1 puro CMV TagRFP, pLKO. 1 puro CMV TagFP635, pLKO. puro UbC TurboGFP, pLKO. L uro UbC TagFP635, pLKO uro IPTG-1xLacO, pLKO uro IPTG-3xLacO, pLP1, pLP2, pLP/VSV-G, pENTR/U6 PLenti6/BLOCK iT-DEST, pLenti6 GW/U6 laminshrna, pcDNA1, 2/V5-GW/lacZ, pLenti6.2/N Lumio/V5-DEST, pGCSIL-GFP, and Lenti 6.2/N Lumio/V5-GW/lacZ.

Preferably, the carrier also includes one or more regulatory elements.

Preferably, the regulatory elements include promoters, enhancers, ribosome binding sites for translation initiation, terminators, polyadenylate sequences, and screening marker genes.

Preferably, the promoter is an inducible promoter, a constitutive promoter, a tissue-specific promoter, a suicidal promoter, or any combination thereof

Host Cell

On the other hand, the present disclosure provides a host cell comprising one or more of the aforementioned peptide, tumor antigen binding peptide, DNA molecule encoding KHL peptide, DNA molecule encoding tumor antigen binding peptide, and the aforementioned carriers.

In one embodiment, the host cell includes one or more of Escherichia coli, Streptomyces, Agrobacterium, yeast cells, plant cells, animal cells, or viruses.

In one embodiment, the virus is a lentivirus.

In one embodiment, the animal cells are human cells.

Preferably, the host cell is an immune cell.

Preferably, the immune cells include one or more of T cells, B cells, K cells, and NK cells.

Preferably, the immune cells are NK cells.

Preferably, the immune cells are autologous or allogeneic.

Preferably, the immune cells are commercialized cell lines. Preferably, the cell line includes NK-92, NKG, YT, NK-YS, HANK-1, YTS, or NKL.

The host cell prepared in the specific example of the present disclosure is called TABP-EIC (Tumor Antigen Binding Peptide Engineering Immune Cell).

Pharmaceutical Composition

On the other hand, the present disclosure provides a pharmaceutical composition, which includes one or more of the aforementioned peptide, tumor antigen binding peptide, DNA molecule encoding KHL peptide, DNA molecule encoding tumor antigen binding peptide, carrier, and the aforementioned host cells.

Preferably, the pharmaceutical composition can be tablets (including sugar coated tablets, film coated tablets, sublingual tablets, oral disintegrating tablets, oral tablets, etc.), pills, powders, granules, capsules (including soft capsules and microcapsules), tablets, syrup, liquid, emulsion, suspension, controlled release preparations (such as instant release preparations, sustained-release preparations, sustained-release microcapsules), aerosols, etc, membranes (such as oral disintegrating films, oral mucosal adhesive films), injections (such as subcutaneous injection, intravenous injection, intramuscular injection, intraperitoneal injection), intravenous drops, transdermal absorption preparations, ointments, lotions, adhesive preparations, suppositories (such as rectal suppositories, vaginal suppositories), small pills, nasal preparations, lung preparations (inhalers), eye drops, etc, oral or parenteral preparations (such as intravenous, intramuscular, subcutaneous, organ, nasal, intradermal, intravenous, intracerebral, rectal, etc.) are administered to the vicinity of the tumor and directly to the lesion.

Preferably, the pharmaceutical composition also includes any pharmaceutically acceptable immune modulators.

Preferably, the immune modulators may include but are not limited to cytokines, chemokines, stem cell growth factors, lymphotoxins, hematopoietic factors, colony stimulating factors (CSF), erythropoietin, thrombopoietin, tumor necrosis factor-a (TNF), TNF-i3, granulocyte colony stimulating factor (G-CSF), granulocyte macrophage colony stimulating factor (GM-CSF), interferon-α, Interferon-β, Interferon-γ, Interferon_λ, Stem cell growth factor, human growth hormone, N-methylthionyl human growth hormone, bovine growth hormone, parathyroid hormone, thyroid hormone, insulin, proinsulin, relaxin, relaxin, follicle stimulating hormone (FSH), thyroid stimulating hormone (TSH), luteinizing hormone (LH), liver growth factor, prostaglandin, fibroblast growth factor, prolactin, placental prolactin, OB protein, designated as “S1 factor” mullerian inhibiting substance, mouse gonadotropin related peptide, inhibin, activator, vascular endothelial growth factor, integrin, NGF-β, Platelet growth factor, TGF-a, TGF-f3, insulin-like growth factor-1, insulin-like growth factor-II, macrophage 43, IL-1, IL-la, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, or lymphotoxin.

Method

On the other hand, the present disclosure provides a method for preparing the aforementioned host cell, which includes the steps of introducing the DNA molecule encoding the KHL peptide, the DNA molecule encoding the tumor antigen binding peptide, or the carrier into the cell.

Preferably, the method of introducing cells can be gene gun method, electroporation method, virus transduction method, or heat shock method.

Preferably, the virus transduction method includes the steps of preparing a recombinant lentivirus and infecting cells with the recombinant lentivirus.

Preferably, the recombinant lentivirus is obtained by transfecting the recombinant lentivirus vector into lentivirus packaging cells and then conducting cell culture;

On the other hand, the present disclosure provides a method for detecting PSMA, which includes the step of contacting a conjugate of the aforementioned KHL peptide with the sample to be tested.

Preferably, the method also includes the step of processing the sample.

Preferably, the detection is non diagnostic.

Preferably, the sample to be tested is suspected to contain PSMA.

Test Kit

On the other hand, the present disclosure provides a test kit for detecting PSMA, which includes reagents used in the aforementioned method for detecting PSMA;

Preferably, the test kit also includes instruments or devices required for detecting PSMA.

Application

On the other hand, the present disclosure provides the application of the aforementioned peptides, tumor antigen binding peptides, DNA molecules encoding KHL peptides, DNA molecules encoding tumor antigen binding peptides, or carriers or host cells in the preparation of the aforementioned pharmaceutical compositions.

On the other hand, the present disclosure provides the application of the aforementioned peptides, tumor antigen binding peptides, DNA molecules encoding KHL peptides, DNA molecules encoding tumor antigen binding peptides, or carriers in the preparation of the aforementioned host cells.

On the other hand, the present disclosure provides the application of DNA molecules encoding KHL peptides and tumor antigen binding peptides in the preparation of the aforementioned carriers.

On the other hand, the present disclosure provides the application of the peptide according to claim 1, the tumor antigen binding peptide according to claim 2, the DNA molecule according to claim 3, the carrier according to claim 4, the host cell according to claim 5, or the pharmaceutical composition according to claim 6 in the preparation of drugs for treating cancer.

Preferably, the cancer is prostate cancer;

On the other hand, the present disclosure provides the application of the aforementioned peptides, tumor antigen-binding peptides, DNA molecules encoding KHL peptides, DNA molecules encoding tumor antigen-binding peptides, carriers or host cells or pharmaceutical compositions in the preparation of test kits for cancer diagnosis.

Preferably, the cancer is prostate cancer.

Preferably, the diagnosis is the detected PSMA.

Preferably, the test kit is the aforementioned test kit.

General Definition:

Unless otherwise defined, the technical and scientific terms used in this article have the same meanings as those commonly understood by one of the ordinary technical personnel in the field.

The term “polynucleotide”, “nucleic acid”, and “oligonucleotide” are interchangeable and refer to the aggregated form of nucleotides of any length, namely deoxyribonucleotides or ribonucleotides or their analogues. Polynucleotides can be further modified after polymerization, for example by conjugating with labeled components. This term also refers to double stranded and single stranded molecules.

As used in this article, the term “carrier” refers to a non chromosomal nucleic acid containing a complete replicon, allowing the carrier to be replicated when placed within the allowed cell, such as through a transformation process. The carrier can replicate in one cell type (such as bacteria), but its ability to replicate in another cell type (such as mammalian cells) is limited. The carrier can be viral or non viral. Example non viral vectors for delivering nucleic acids include exposed DNA; DNA compounded with cationic lipids, either alone or in combination with cationic polymers; Anionic and cationic liposomes; DNA-protein complexes and particles containing DNA condensed with cationic polymers such as heteropoly lysine, fixed length oligopeptides, and polyethylene imines, in some cases also included in liposomes;

As used in this article, the terms “peptide”, “polypeptide”, and “protein” are interchangeable and refer to compounds with amino acid residues covalently linked by peptide bonds.

The term “expression” or “expression” refers to the production of gene products in cells.

### BRIEF DESCRIPTION OF DRAWINGS

FIG. 1 shows the immunofluorescence results of Lncap cell lines with high levels of peptide KHL and PSMA expression.

FIG. 2 shows the immunofluorescence results of PC3 cell lines with low levels of peptide KHL and PSMA expression.

FIG. 3 shows the fluorescence detection of mice on the 14th day; A is the blank control, B is the control group, TABP-EIC-GTI cells, C is the experimental group, TABP-EIC-KHL.

FIG. 4 shows the fluorescence detection of mice on the 28th day; A is the blank control, B is the control group, TABP-EIC-GTI cells, C is the experimental group, TABP-EIC-KHL.

FIG. 5 shows the fluorescence detection of mice on the 42nd day; A is the blank control, B is the control group, TABP-EIC-GTI cells, C is the experimental group, TABP-EIC-KHL.

### DETAILED DESCRIPTION

The following is a further explanation of the present disclosure in conjunction with embodiments. The following is only a preferred embodiment of the present disclosure and does not impose any other form of limitation on the present disclosure. Any technical personnel familiar with the profession may use the disclosed technical content to modify it into equivalent embodiments with the same changes. Any simple modifications or equivalent changes made to the following embodiments based on the technical essence of the present disclosure without departing from the content of the present disclosure scheme shall fall within the scope of protection of the present disclosure.

### Example 1: Peptide Library Screening

1. Experimental Purpose

The present disclosure uses the Ph.D.-12 phage display peptide library kit to screen peptide KHL that specifically binds to PSMA.

2. Composition of Phage Display Peptide Library Kit for Ph.D.-12

Random twelve peptide phage display library: 100 μL. 1.5×10¹³ pfu/mL, stored in TBS solution containing 50% glycerol, complexity-2.7×10⁹ transformants-28gIII sequencing primers: 5′-HOGATTGGGATTTGCTAACAAC-3′, 100 pmol, 1 pmol/μL-96 g III sequencing primer: 5′-HOCCTCATATAGTTAGCGTAGCGTAACG-3′, 100 pmol/1 pmol/μL; E. Coli ER2738 Host bacterium F′laclq Δ(lacZ) M15 proA+B+zzf:: Tn10 (TetR)/fhuA2 supE thi Δ(lac proAB) Δ(hsdMS mcrB) 5 (rk mk McrBC−): This strain is provided in the form of a bacterial culture containing 50% glycerol, non receptive cells, and stored at −70° C.; Streptavidin, freeze-dried powder 1.5 mg; Biotin: 10 mM 100 μL.

3. Experimental methods.

First Day

Depending on the number and types of target molecules that need to be simultaneously selected for library selection, the selection experiment is conducted on a single sterilized polystyrene culture dish, 12 or 24 well plates, and 96 well microplates. Each target molecule is coated with at least one plate (or well), and the amount given in the following method is the amount of the 60×15 mm culture dish, the content in parentheses is the amount of the plate, and the dosage of microplate is adjusted accordingly for other medium-sized wells. However, in each case, the number of added bacteriophages is the same: 1.5×10¹¹ virion.

(1) 100 μg/mL target molecule solution (soluble in 0.1M NaHCO₃ at pH 8.6) was prepared, if stable target molecules are required, other buffer solutions with similar ionic strength (including metal ions, etc.) can also be used.

(2) 1.5 mL above solution per plate (well) (each well of the microporous plate is 150 μL) was added, rotate the repeatedly until the surface is completely wet.

(3) Slightly shaked at 4° C. in a humidified container (such as a sealable plastic box lined with wet tissue), incubated overnight, and stored the tablet in this container at 4° C. for future use.

The Second Day

(4) Selected ER2738 monoclonal antibody (plate laid during phage titer measurement) and placed it in 10 mL LB liquid culture medium. If the washed phage is amplified on the same day, ER2738 can also be inoculated into 20 mL LB liquid culture medium and cultured in a 250 mL triangular flask at 37° C. under intense shaking.

(5) Poured out the coating liquid from each plate, placed the plate upside down on a clean tissue and vigorously pat to remove residual solution. Fill each plate (or hole) with sealing liquid and let it sit at 4° C. for at least 1 hour.

(6) Washed the plate 6 times, rotating each time to ensure that the bottom and edges of the plate or hole are washed. Poured out the buffer solution, inverted it on a clean tissue, and pat it to remove residual solution (or use an automatic plate washer).

(7) Used 1 mL (for microporous plates, use 100 u L) dilution of TBST buffer 4×10¹⁰ phages (i.e. 10 μL of the original library, and then add it to the coated plate, gently shake at room temperature for 10-60 minutes.

(8) Dumped to remove unbound bacteriophages, inverted the plate and pat it on a clean tissue to remove residual solution.

(9) Washed the plate with TB ST buffer solution 10 times according to the method described in 6, and replaced it with a clean tissue each time to avoid cross contamination;

(10) Based on the intermolecular interactions studied, 1 mL (100 for microporous plates) was used μL) Appropriate elution buffer is used to elute the bound phage, and the known ligand of the target molecule is dissolved in TBS solution at a concentration of 0.1-1 mM or in a free target molecule solution (˜100 μ G/mL dissolved in TBS) competitively elute the bound phage from the fixed target molecule, gently shake at room temperature for 10-60 minutes, and inhale the eluent into another clean micro centrifuge tube; Non specific buffer solutions such as 0.2M Glycine HCl (pH 2.2) and 1 mg/mL BSA can also be used to separate bound molecules: gently shake for >10 minutes, and the eluent is sucked into another clean micro centrifuge tube, followed by 150% centrifugation μL (for micro pores, use 15 μ L) Neutralize the above eluent with 1M Tris HCl (pH 9.1);

(11) Measured a small amount (˜1 μL) according to the routine M13 method described above. The titer of the eluate can be sequenced if necessary, using the phage obtained from the first or second round of eluate titer determination. The method is as follows: if necessary, the remaining eluate can be stored at 4° C. overnight and amplified the next day. At this time, ER2738 can be cultured overnight in LB Tet medium. The second day, the culture can be diluted 1:100 in 20 mL LB (packed in a 250 mL triangular flask), and the unamplified eluate can be added. The culture can be vigorously shaken at 37° C. for 4.5 hours, continue with step 13.

(12) Amplified the remaining eluate: added the eluate to 20 mL of ER2738 culture (the bacterial body is in the pre logarithmic stage), Shaked vigorously at 37° C., and incubated for 4.5 hours.

(13) Transferred the culture into a centrifuge tube and centrifuge at 4° C. at 10,000 rpm for 10 minutes. Transferred the supernatant into another centrifuge tube and centrifuged again;

(14) Transferred the upper 80% of the supernatant into a fresh tube, added 1/6 volume of PEG/NaCl, and allowed the bacteriophage to settle at 4° C. for at least 60 minutes, overnight;

The Third Day

(15) Centrifuged PEG at 4° C. and 10,000 rpm for 15 minutes, poured out the supernatant, then centrifuged briefly to remove the remaining supernatant.

(16) The sediment was resuspended in 1 mL TBS, and the suspension was transferred to a micro centrifuge tube. The residual cells were precipitated by centrifugation at 4° C. for 5 minutes.

(17) Transferred the supernatant into another fresh microcentrifuge tube, precipitated with 1/6 volume of PEG/NaCl, incubated on ice for 15-60 minutes, centrifuged at 4° C. for 10 minutes, discarded the supernatant, briefly centrifuged again, and used a micropipette to remove the remaining supernatant.

(18) Sediment resuspended in 200 μL TBS, 0.02% NaN₃, centrifuged for 1 minute, precipitated any remaining insoluble matter, and transferred the supernatant into a fresh tube, which is the eluted product after amplification.

(19) According to the conventional M13 method mentioned above, the elute amplified by LB/IPTG/Xgal plate titration was stored at 4° C.

(20) Coated another plate or hole for the second round of selection.

The Fourth Day and the Fifth Day

(21) Determine the titer based on the number of blue spots on the counting plate, and use this value to calculate the addition amount corresponding to 1-2×10¹¹ pfu. If the titer is too low, the next few rounds of panning can be tested with bacteriophage addition amounts as low as 10⁹ pfu.

(22) Conducted the second round of panning: 1-2×10¹¹ pfu of the elutriates amplified from the first round of panning is repeated for steps 4-18, and increased the concentration of Tween to 0.5% (v/v) during the cleaning step.

(23) Measured the titer of the elutriate obtained from the second round of elution amplification on LB/IPTG/Xgal plates.

(24) Coated another plate or hole for the third round of panning.

The Sixth Day

(25) Conducted the third round of panning: 2×10¹¹ pfu of bacteriophage in the elutriates amplified from the second round of panning was used to repeat steps 4-11, and used 0.5% (v/v) Tween in the cleaning step.

(26) Measured the titer of the elutriates obtained from the third round of elution on LB/IPTG/Xgal plates without amplification. The elutriates from the third round do not need to be further amplified, unless a fourth round of elution is required. The bacteriophages obtained during the titer determination can be used for sequencing: as long as the plate culture time does not exceed 18 hours, it is easy to be missing if the culture time is too long, and the remaining elutes are stored at 4° C.

(27) Selected an ER2738 monoclonal and cultured it overnight in LB Tet medium.

4. Experimental Results

The experimental results showed that the peptide KHL specifically binding to PSMA obtained through screening had an amino acid sequence of KHLHYHSSVRYG (SEQ ID NO: 1).

### Example 2: Verification of KHL Peptide Specificity

Immunofluorescence detection was performed on KHL peptides in Lncap cell lines with high PSMA expression and PC3 cell lines with low PSMA expression, using FITC as the fluorescent marker.

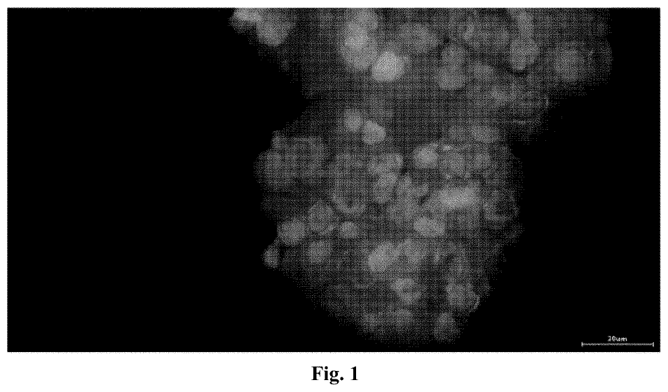

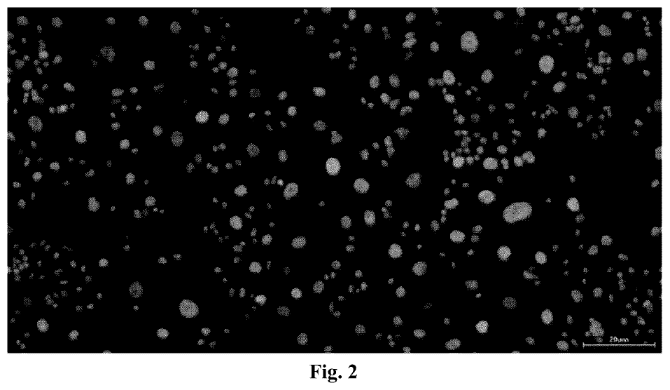

The fluorescence detection of KHL peptide and Lncap cell line is shown in FIG. 1, and the fluorescence detection of KHL peptide and PC3 cell line is shown in FIG. 2.

In FIG. 1, the center of the dot is the nucleus, and the light color around the dot is the fluorescence displayed by KHL binding to the cell surface antigen PSMA. In FIG. 2, there is only staining of the nucleus. The cell surface fluorescence labeled by KHL only appears in FIG. 1, indicating that the binding of KHL to the cell surface antigen PSMA has high specificity.

### Example 3: Validation of the Inhibitory Effect of TABP-EIC-KHL on Tumors

1. Preparation of NK Cells

The NK cells used in this patent experiment are all derived from peripheral blood mononuclear cells (PBMC) amplification.

2. Construction of tumor antigen binding peptide expression vector

The structural sequences of tumor antigen binding peptides were obtained through gene synthesis (universal biology), and the expression vector was pLenti EFla Backbone (NN) (add gene #27961). The insertion sites of tumor antigen binding peptide structures are BsiWI and EcoRI.

3. Lentivirus packaging

Mixed the TABP-EIC skeleton vector and auxiliary vectors pMD2. G (additive #12259), pMDLg/pRRE (additive #12251), and pRSV Rev (additive #12253) in a ratio of 10:5:3:2, and transfected 293T cells with a 20 μg plasmid every 10 ml of the transfection system. After 48 hours and 72 hours, collected the supernatant, purified and concentrated it to obtain the lentivirus.

4. Lentivirus transduction

Mixed the concentrated lentivirus with NK cells purified at a rate of 200 ul per 1 million cells, and then cultured it in a 37° C. incubator with 5% CO₂. After 24 hours, completely changed the solution.

5. Amplification of TABP-EIC-GTL cells

The TABP-EIC cells obtained after infection with lentivirus were cultured and expanded normally.

6. Detection of the expression efficiency of tumor antigen binding peptides in TABP-EIC-GTL cells

On the seventh day after infection with lentivirus in TABP-EIC cells, a portion of the cells were taken to extract the genome for RT-PCR detection.

7. Experimental results

According to the RT-PCR results, according to the formula, the infection efficiency (%)=63.21−6.36×ΔCT (detection group CT−control group CT) generally requires an infection rate of over 20% before use.

The KHL sequence in the tumor antigen binding peptide structure can have multiple replicates, with three replicates in this example. The amino acid sequence of the tumor antigen binding peptide is shown in SEQ ID NO: 5, and the nucleic acid sequence of the tumor antigen binding peptide is shown in SEQ ID NO: 10.

GFP labeled prostate cancer cell line (Lncap GFP) was used to induce intraperitoneal tumor formation in mice. On the 14th day of tumor formation, intraperitoneal perfusion was performed, followed by blank control (physiological saline), control group (TABP-EIC-GTI cells), and TABP-EIC-KHL cells. The intraperitoneal perfusion was performed once a week for 3 weeks at a rate of 5 million cells per mouse.

On days 14, 28, and 42, fluorescence imaging was performed on mice to observe tumor size.

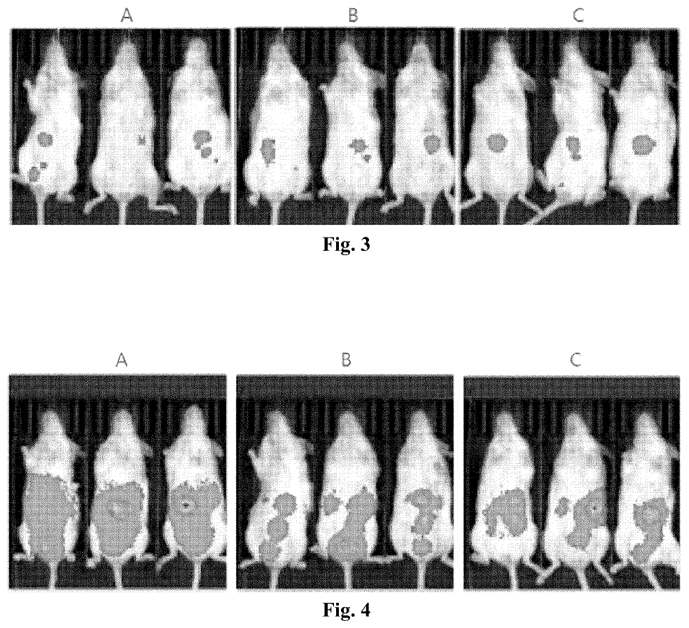

On the 14th day of fluorescence imaging, as shown in FIG. 3, A is the blank control group, B is the control group (TABP-EIC-GTI cells), and C is TABP-EIC-KHL.

On the 28th day of fluorescence imaging, as shown in FIG. 4, A is the blank control group, B is the control group (TABP-EIC-GTI cells), and C is TABP-EIC-KHL.

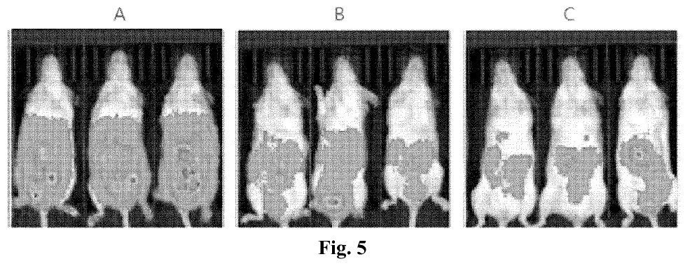

On the 42nd day of fluorescence imaging, as shown in FIG. 5, A is the blank control group, B is the control group (TABP-EIC-GTI cells), and C is TABP-EIC-KHL.

This experiment demonstrates that both TABP-EIC-GTL and TABP-EIC-KHL cells have significant inhibitory effects on tumors, and TABP-EIC-KHL has a better therapeutic effect.

1. A KHL peptide or its conjugate specifically binding to PSMA, wherein the KHL peptide is selected from any of the following:
1) peptide shown in SEQ ID NO:1;
2) derived peptides that are formed by replacing, missing, or adding one or more amino acid residues to the peptide shown in SEQ ID NO:1 and have essentially the same function; and
3) peptides with over 90% homology with the peptide shown in SEQ ID NO:1, specifically, the peptide has 90%, 91%, 92%, 93%, 94%, 95%, 96%, 97%, 98%, 99% homology with SEQ ID NO:1;
wherein, the KHL peptide is a peptide shown in SEQ ID NO:1;
wherein, the conjugate of the KHL peptide includes a KHL peptide and a detectable marker;
wherein, the detectable markers include one or more of a fluorescent dye, a fluorescent molecule, a chemiluminescent marker, a dye molecule, a phosphorescent molecule, a biotin, a radioactive isotope, a molecule that can absorb in a UV spectrum, a molecule that can absorb in near-infrared radiation, or a molecule that can absorb in far-infrared radiation;
wherein, the detectable marker is a fluorescent molecule; and
wherein, the fluorescent molecule is FITC.

2. A tumor antigen binding peptide, wherein the tumor antigen binding peptide comprises a specific binding region specifically binding to PSMA;
wherein, the specific binding region includes the KHL peptide according to claim 1;
wherein, the specific binding region is three replicates of the KHL peptide according to claim 1;
wherein, the three replicates of the KHL peptide are connected by GGGS;
wherein, the tumor antigen binding peptide further includes a hinge area, a transmembrane domain, and/or a signal transduction domain;
wherein, the hinge area selects CD8 a hinge area;
wherein, the amino acid sequence of the hinge area is shown in SEQ ID NO:11;
wherein, the transmembrane domain selects a transmembrane region of a 2B4 gene;
wherein, the amino acid sequence of the transmembrane domain is shown in SEQ ID NO:2;
wherein, the signal transduction domain includes a co stimulus domain and/or a primary signal transduction domain;
wherein, the co stimulus signal selects an intracellular signal transduction structure of the 2B4 gene;
wherein, the amino acid sequence of the co stimulus signal is shown in SEQ ID NO:3;
wherein, the primary signal transduction domain selects the intracellular signal transduction structure of a NKG2D gene;
wherein, the amino acid sequence of the primary signal transduction domain is shown in SEQ ID NO:4;
wherein, the composition sequence of the tumor antigen binding peptide is KHL peptide-hinge area-transmembrane domain-co stimulatory domain-primary signal transduction domain;
wherein, the amino acid sequence of the tumor antigen binding peptide is at positions 22-316 as shown in SEQ ID NO:5;
wherein, the tumor antigen binding peptide can also be connected to a signal peptide; and
wherein, the amino acid sequence of the tumor antigen binding peptide connected to the signal peptide is shown in SEQ ID NO:5.

3. An application of a KHL peptide or its conjugate specifically binding to PSMA, or a tumor antigen binding peptide,
wherein the KHL peptide is selected from any of the following:
1) peptide shown in SEQ ID NO:1;
2) derived peptides that are formed by replacing, missing, or adding one or more amino acid residues to the peptide shown in SEQ ID NO:1 and have essentially the same function;
3) peptides with over 90% homology with the peptide shown in SEQ ID NO:1, specifically, the peptide has 90%, 91%, 92%, 93%, 94%, 95%, 96%, 97%, 98%, 99% homology with SEQ ID NO:1;
wherein, the KHL peptide is a peptide shown in SEQ ID NO:1;
wherein, the conjugate of the KHL peptide includes a KHL peptide and a detectable marker;
wherein, the detectable markers include one or more of a fluorescent dye, a fluorescent molecule, a chemiluminescent marker, a dye molecule, a phosphorescent molecule, a biotin, a radioactive isotope, a molecule that can absorb in the UV spectrum, a molecule that can absorb in near-infrared radiation, or a molecule that can absorb in far-infrared radiation;
wherein, the detectable marker is a fluorescent molecule;
wherein, the fluorescent molecule is FITC;
wherein the tumor antigen binding peptide comprises a specific binding region specifically binding to PSMA;
wherein, the specific binding region includes the KHL peptide according to claim 1;
wherein, the specific binding region is three replicates of the KHL peptide according to claim 1;
wherein, the three replicates of the KHL peptide are connected by GGGS;
wherein, the tumor antigen binding peptide further includes a hinge area, a transmembrane domain, and/or a signal transduction domain;
wherein, the hinge area selects CD8 a hinge area;
wherein, the amino acid sequence of the hinge area is shown in SEQ ID NO:11;
wherein, the transmembrane domain selects a transmembrane region of a 2B4 gene;
wherein, the amino acid sequence of the transmembrane domain is shown in SEQ ID NO:2;
wherein, the signal transduction domain includes a co stimulus domain and/or a primary signal transduction domain;
wherein, the co stimulus signal selects an intracellular signal transduction structure of the 2B4 gene;
wherein, the amino acid sequence of the co stimulus signal is shown in SEQ ID NO:3;
wherein, the primary signal transduction domain selects the intracellular signal transduction structure of the NKG2D gene;
wherein, the amino acid sequence of the primary signal transduction domain is shown in SEQ ID NO:4;
wherein, the composition sequence of the tumor antigen binding peptide is KHL peptide-hinge area-transmembrane domain-co stimulatory domain-primary signal transduction domain;
wherein, the amino acid sequence of the tumor antigen binding peptide is at positions 22-316 as shown in SEQ ID NO:5;
wherein, the tumor antigen binding peptide can also be connected to a signal peptide; and
wherein, the amino acid sequence of the tumor antigen binding peptide connected to the signal peptide is shown in SEQ ID NO:5;
wherein the application comprises employing the KHL peptide or its conjugate specifically binding to PSMA or the tumor antigen binding peptide as a component of a DNA molecule, a carrier, a host cell, a pharmaceutical composition, a component for a method for detecting PSMA, a component of a test kit for detecting PSMA.

4. The application of claim 3, wherein the DNA molecule encodes the KHL polypeptide, the tumor antigen binding peptide, or the tumor antigen binding peptide with a signal peptide;
wherein, the sequence of the DNA molecule encoding the KHL polypeptide is shown in SEQ ID NO:6;
wherein, the sequence of the DNA molecule encoding the tumor antigen binding peptide is a 64th-948th position of SEQ ID NO:10; and
wherein, the sequence of the DNA molecule encoding the tumor antigen binding peptide connected with the signal peptide is shown as SEQ ID NO:10.

5. The application of claim 3, wherein the carrier contains the DNA molecule;
wherein, the carrier includes a plasmid, a lentiviral vector, an adenovirus vector, or a retrovirus vector; and
wherein, the carrier also includes one or more regulatory elements.

6. The application of claim 3, wherein the host cell comprises one or more of the peptides, the tumor antigen binding peptides, the DNA molecules, and the carriers;
wherein, the host cell includes Escherichia coli, Streptomyces, Agrobacterium, yeast cells, plant cells, animal cells or viruses;
wherein, the viruses are lentiviruses;
wherein, the animal cells are human immune cells; and
wherein, the immune cells are NK cells.

7. The application of claim 3, wherein the pharmaceutical composition comprises one or more of the peptides, the tumor antigen binding peptide, the DNA molecule, the carrier, and the host cell;
wherein, the pharmaceutical composition also comprises any pharmaceutically acceptable immune modulators.

8. The application of claim 3, wherein the method for detecting PSMA comprises the step of contacting a conjugate of the KHL peptide with a sample to be tested;
wherein, the detection is non diagnostic; and
wherein, the sample to be tested is suspected to contain PSMA.

9. The application of claim 3, wherein the test kit for detecting PSMA comprises the reagent used in the method for detecting PSMA;
wherein, the test kit also includes instruments or devices required for detecting PSMA.

10. The application of claim 3, wherein the application further includes any one of the following:
1) the application of the peptide, the tumor antigen binding peptide, the DNA molecule, the carrier, or the host cell in the preparation of the drug composition;
2) the application of the peptide, the tumor antigen binding peptide, the DNA molecule, or the carrier in the preparation of the host cell;
3) the application of the DNA molecule in the preparation of the carrier;
4) the application of the peptide, the tumor antigen binding peptide, the DNA molecule, the carrier, the host cell, or the drug composition in the preparation of drugs for treating cancer;
wherein, the cancer is prostate cancer; and
5) the application of the peptide conjugate in the preparation of a test kit for diagnosis of cancer;
wherein, the cancer is prostate cancer;
wherein, the diagnosis is the detected PSMA; and
wherein, the test kit is the test kit for detecting PSMA.

11. The application of claim 10, wherein the application comprises a method for preparing the host cell, which comprises the steps of introducing one or more DNA molecules encoding the peptide, the tumor antigen binding peptide, the DNA molecule, and the carriers into the host cell;
wherein, the method of introducing into host cell can be gene gun method, electroporation method, virus transduction method, or heat shock method.
